(19) United States
(12) Patent Application Publication
(10) Pub. No.: US 2024/0331805 A1
(43) Pub. Date: Oct. 3, 2024

(54) IDENTIFICATION OF AMINO ACID ISOMERS USING DIAGNOSTIC FRAGMENT IONS IN MS/MS DATA

(71) Applicant: DH Technologies Development Pte. Ltd., Singapore (SG)

(72) Inventors: Armandine BOUDREAU, Aurora (CA); Mona HAMADA, Concord (CA); Stephen SCIUTO, Toronto (CA)

(21) Appl. No.: 18/588,316

(22) Filed: Feb. 27, 2024

Related U.S. Application Data
(60) Provisional application No. 63/487,933, filed on Mar. 2, 2023.

(51) Int. Cl. G16B 40/10

(52) U.S. Cl. CPC G16B 40/10

(57) ABSTRACT

Computer-implemented methods and non-transitory computer readable storage media for isobaric amino acid differentiation, where an MS/MS data set may be processed, wherein the processing includes determining whether a protein sample comprises at least one isobaric amino acid, analyzing whether any diagnostic fragment ions of the at least one isobaric amino acid are present in the protein sample, assigning a score to the at least one isobaric amino acid based on the diagnostic fragment ions present in the MS/MS data set, evaluating the identity of the at least one isobaric amino acid present in the protein sample based on the assigned score, and reporting the evidence of the identity of the at least one isobaric amino acid present in the protein sample.

### RELATED APPLICATIONS

The present patent application claims the priority benefit of U.S. Provisional Patent Application Ser. No. 63/487,933, filed Mar. 2, 2023, the content of which is hereby incorporated by reference in its entirety into this disclosure.

### BACKGROUND

Mass spectrometry (MS) is an analytical technique used for the detection and quantitation of chemical compounds. MS involves ionizing compounds and analyzing the mass-to-charge (m/z) ratios of the ions with a detector. One of ordinary skill in the art understands that a mass of the molecule can be found from an m/z by multiplying the m/z by the absolute value of the charge and taking into account the mass of the charge carrier. Similarly, the m/z can be found from a mass of the molecule and charge carrier by dividing the mass by the charge.

An MS scan includes the selection of a precursor ion or precursor ion range. In some cases, tandem mass spectrometry (also known as MS/MS) can be employed to aid in the structure elucidation of certain peaks from the MS scan. Tandem mass spectrometry or MS/MS involves the ionization of one or more compounds of interest from a sample, selection of one or more precursor ions of the one or more compounds, fragmentation of the one or more precursor ions into product ions, and mass analysis of the product ions. Through this fragmentation and subsequent mass analysis, chemists are able to utilize MS/MS to aid in compound structure determination. Further, both MS and MS/MS data may be collected in both polarity modes on the mass spectrometer (i.e., positive and negative).

Mass spectrometry and MS/MS are utilized in the structure elucidation of protein and peptide samples. Isobaric amino acids are amino acids that have identical molecular weights. Two or more amino acids with identical molecular weights may also be referred to more generally as isomers. Structure elucidation of protein and peptide samples that contain isobaric amino acid residues present a challenge in differentiating between isobaric amino acids using mass spectrometric techniques. Therefore, there exists a need for techniques to differentiate between isobaric amino acids using mass spectrometry.

### SUMMARY

In various aspects, a computer-implemented method includes processing, with one or more computing devices, an electron-based dissociation (ExD) MS/MS data set of a protein sample, the MS/MS data set comprising a spectrum with mass-to-charge ratio (m/z) peaks that correspond to fragment ions. The processing further includes determining whether the protein sample comprises at least one isobaric amino acid; analyzing whether any diagnostic fragment ions of the at least one isobaric amino acid are present in the protein sample; assigning a score to the at least one isobaric amino acid based on the diagnostic fragment ions present in the MS/MS data set; evaluating an identity of the at least one isobaric amino acid present in the protein sample based on the assigned score; and reporting the evidence of the identity of the at least one isobaric amino acid present in the protein sample.

In certain aspects, the assigning a score further includes assigning independently weighted values for each diagnostic fragment ion.

In certain aspects, the score further includes assigning a higher weight when a larger number of diagnostic fragment ions are present.

In certain aspects, the score further includes assigning higher weight to a complementary ion pair than a non-complementary ion pair.

In certain aspects, the reporting includes displaying a first indicator or a second indicator when one or more diagnostic fragment ions are present in the MS/MS data set, wherein the first indicator indicates the score is above a score threshold and the second indicator indicates the score is below a score threshold.

In certain aspects, the reporting further includes displaying any diagnostic fragment ions that are present in the MS/MS data set.

In certain aspects, the at least one isobaric amino acid includes isoaspartic acid. In further aspects, the diagnostic fragment ions for isoaspartic acid are c′ₙ, c′ₙ+57, z′ₘ, and z′ₘ−57. In further aspects, the z′ₘ and z′ₘ−57 diagnostic fragment ions are a first complementary ion pair and c′ₙ and c′ₙ+57 diagnostic fragment ions are a second complementary ion pair.

In certain aspects, the at least one isobaric amino acid includes isoleucine. In further aspects, the diagnostic fragment ions for isoleucine are z′ₘ, wₘ (z′ₘ−15), and wₘ (z′ₘ−29).

In certain aspects, the at least one isobaric amino acid includes leucine. In further aspects, the diagnostic fragment ions for leucine are z′ₘ and wₘ (z′ₘ−43).

In certain aspects, the computer-implemented method is incorporated into a peptide mapping workflow.

In other aspects, one or more non-transitory computer-readable storage media comprise instructions, which when executed by one or more computing devices, cause the one or more computing devices to process, with one or more computing devices, an electron-based dissociation (ExD) MS/MS data set of a protein sample, the MS/MS data set comprising a spectrum with mass-to-charge ratio (m/z) peaks that correspond to fragment ions. The processing may further include determining whether the protein sample comprises at least one isobaric amino acid; analyzing whether any diagnostic fragment ions of the at least one isobaric amino acid are present in the protein sample; assigning a score to the at least one isobaric amino acid based on the diagnostic fragment ions present in the MS/MS data set; evaluating an identity of the at least one isobaric amino acid is present in the protein sample based on the assigned score; and reporting the evidence of the identity of the at least one isobaric amino acid present in the protein sample.

### DESCRIPTION OF DRAWINGS

Various aspects and embodiments of the present disclosure are shown in the drawings and described therein and elsewhere throughout the disclosure. In the drawings, like references indicate like parts.

FIG. 1 is a schematic diagram of a mass spectrometer system, in accordance with an example embodiment of the disclosure.

FIG. 2 is a block diagram that illustrates a computer system, upon which aspects of the present teachings may be implemented.

FIG. 3 is a flowchart of an analytical process which may be implemented by the mass spectrometer system of FIG. 1.

FIG. 4 is a flowchart of a peptide mapping workflow.

FIG. 5 is a flowchart of a middle-down workflow.

FIGS. 6A-6C are illustrations of an electron activated dissocitation (EAD) family. The EAD family is classified by precursor species and the kinetic energy of the electron beam. FIG. 6A illustrates EAD techniques for singly charged cations. FIG. 6B illustrates EAD techniques for protonated peptides and peptides. FIG. 6C illustrates EAD techniques for anions.

### DETAILED DESCRIPTION

Unless otherwise defined, all technical and scientific terms used herein have the same meaning as commonly understood by one of ordinary skill in the art to which the methods described herein belong.

As utilized herein the terms “circuits” and “circuitry” refer to physical electronic components (e.g., hardware), and any software and/or firmware (“code”) that may configure the hardware, be executed by the hardware, and/or otherwise be associated with the hardware. As used herein, for example, a particular processor and memory (e.g., a volatile or non-volatile memory device, a general computer-readable medium, etc.) may comprise a first “circuit” when executing a first one or more lines of code and may comprise a second “circuit” when executing a second one or more lines of code. Additionally, a circuit may comprise analog and/or digital circuitry. Such circuitry, for example, may operate on analog and/or digital signals. It should be understood that a circuit may be in a single device or chip, on a single motherboard, in a single chassis, in a plurality of enclosures at a single geographical location, in a plurality of enclosures distributed over a plurality of geographical locations, etc. Similarly, the term “module”, for example, may refer to a physical electronic components (e.g., hardware) and any software and/or firmware (“code”) that may configure the hardware, be executed by the hardware, and or otherwise be associated with the hardware.

As utilized herein, circuitry or module is “operable” to perform a function whenever the circuitry or module comprises the necessary hardware and code (if any is necessary) to perform the function, regardless of whether the performance of the function is disabled or not enabled (e.g., by a user-configurable setting, factory trim, etc.).

As utilized herein, “and/or” means any one or more of the items in the list joined by “and/or”. As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y, and z.”

As utilized herein, the term “exemplary” means serving as a non-limiting example, instance, or illustration. Further, as utilized herein, the terms “for example” and “e.g.,” set off lists of one or more non-limiting examples, instances, or illustrations.

The terminology used herein is for the purpose of describing particular examples only and is not intended to be limiting of the disclosure. As used herein, the singular forms are intended to include the plural forms as well, unless the context clearly indicates otherwise. It will be further understood that the terms “comprises,” “includes,” “comprising,” “including,” “has,” “have,” “having,” and the like when used in this specification, specify the presence of stated features, integers, steps, operations, elements, and/or components, but do not preclude the presence or addition of one or more other features, integers, steps, operations, elements, components, and/or groups thereof.

It will be understood that, although the terms first, second, etc. may be used herein to describe various elements, these elements should not be limited by these terms. These terms are only used to distinguish one element from another element. Thus, for example, a first element, a first component or a first section discussed below could be termed a second element, a second component or a second section without departing from the teachings of the present disclosure. Similarly, various spatial terms, such as “upper,” “lower,” “side,” and the like, may be used in distinguishing one element from another element in a relative manner. It should be understood, however, that components may be oriented in different manners, for example a semiconductor device may be turned sideways so that its “top” surface is facing horizontally and its “side” surface is facing vertically, without departing from the teachings of the present disclosure.

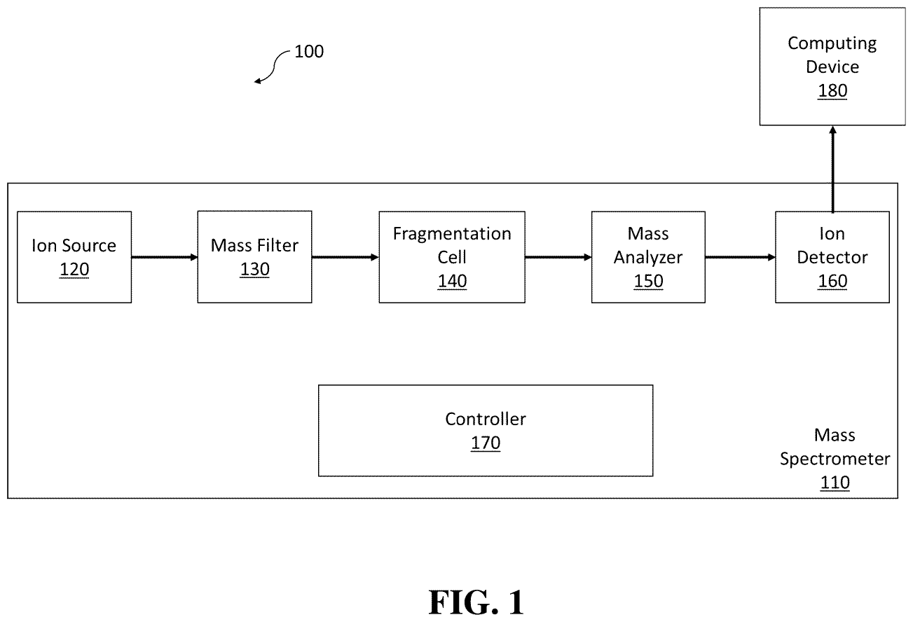

FIG. 1 is a non-limiting example of a mass analysis system 100. As shown, the mass analysis system 100 includes a mass spectrometer 110. The mass spectrometer 110 may include an ion source 120, a mass filter 130, a fragmentation cell 140, a mass analyzer 150, an ion detector 160, and a controller 170. The mass spectrometer 110 may separate, fragment, and detect ions of interest from a given sample. The one or more computing devices 180 may be operative to control the operation of the mass spectrometer 110, receive spectral data generated by the mass spectrometer, and manage the spectral data received from the mass spectrometer 110. Typically, the mass spectrometer 110 may format a detected ion signal generated by the ion detector 160 in spectral data representative of one or more mass spectra. The one or more computing devices 180 may receive the spectral data from the mass spectrometer 110 and analyze the spectral data to produce one or more data reports, graphs, etc.

A mass analyzer 150 may receive the generated ions from the ion source 120 for mass analysis. The mass analyzer 150 may be operative to selectively separate ions of interest from generated ions received from the ion source 120 and to deliver the ions of interest to an ion detector 160 that generates a mass spectrometer signal indicative of detected ions to the one or more computing devices 180. In some aspects, the separate ions of interest may be indicated in an analysis instruction associated with that sample.

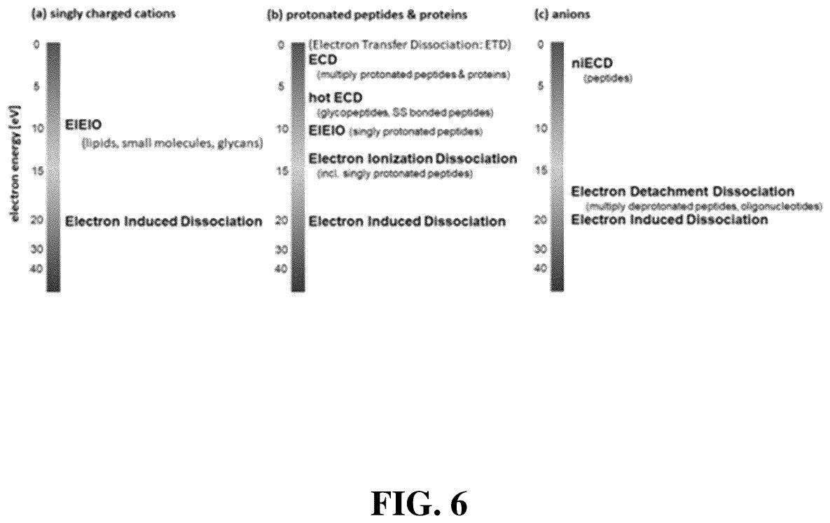

When conducting MS analysis, the mass analyzer 150 may receive the generated ions directly from the ion source. When conducting tandem mass spectrometry analysis (also referred to as MS/MS), the ions generated from the ion source 120 are subjected to fragmentation in a fragmentation cell 140 prior to being received by the mass analyzer 150. The fragmentation cell 140 may be configured to conduct certain fragmentation (also referred to as dissociation) techniques or methodologies. For example, the fragmentation cell 140 may be configured to conduct radical-induced dissociation methodologies, such as electron-based dissociation (ExD). ExD may include, but is not limited to, electron-activated dissociation (EAD) techniques such as electron-induced dissociation (EID), electron impact excitation in organics (EIEIO), electron capture dissociation (ECD), hot ECD, negative ion ECD (niECD), electron detachment dissociation (EDD), and electron transfer dissociation (ETD). FIGS. 6A, 6B, and 6C illustrate various EAD techniques.

As shown, the mass spectrometer 110 may be coupled to one or more computing devices 180. The one or more computing devices 180 may control operation of the mass spectrometer 110, receive spectral data from the mass spectrometer 110, analyze the spectral data, and present results of such analysis of the spectral data.

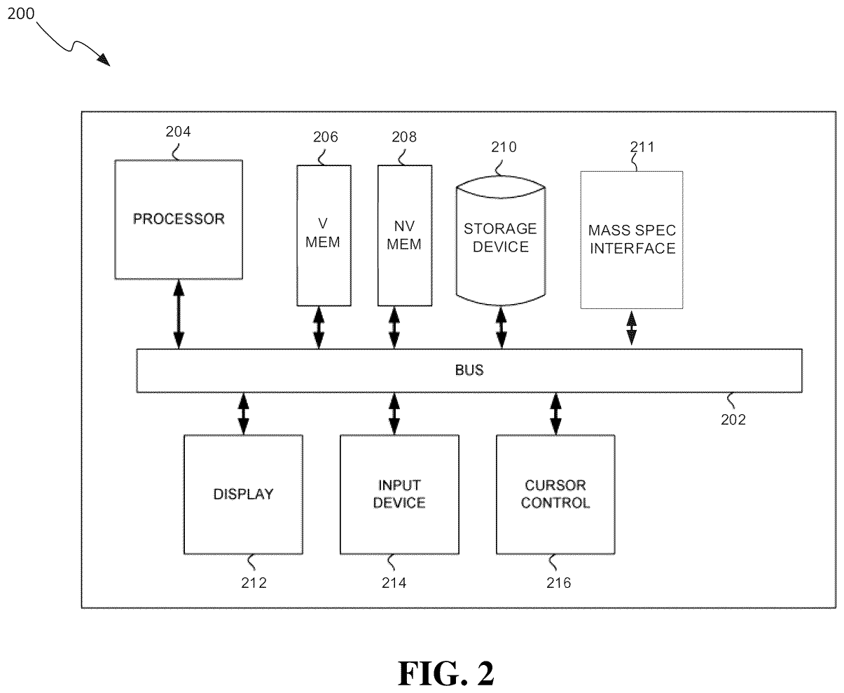

The one or more computing devices 180 may comprise a single computing device or may comprise a plurality of distributed computing devices in operative communication with the mass spectrometer 110 and/or another. FIG. 2 illustrates a high-level block diagram of an example computing device 200 which may implement one or more of the computing devices 180. To this end, the computing device 200 may comprise a bus 202, a processor 204, volatile memory 206, non-volatile storage 208, storage device 210, and a mass spectrometer interface 211. The bus 202 may comprise various signal lines, interfaces, etc., that operatively interconnect components of the computing device 200 such as processor 204, volatile memory 206, non-volatile storage 208, storage device 210, and mass spectrometer interface 211 to permit transfers of information and/or control signals between such components of the computing device 200.

The processor 204 may include a plurality of processing elements or cores, which may be packaged as a single processor or in a distributed arrangement. Furthermore, in some aspects, a plurality of virtual processing elements may be provided to provide the control or management operations for the computing device 200.

The memory 206 may include random access memory (RAM) and/or other dynamic storage devices coupled to bus 202. The memory 206 may store instructions executed by processor 204. The memory 206 may also store temporary variables, intermediate information, and/or other data resulting from the execution of the instructions by processor 204. The non-volatile memory 208 may include read-only-memory (ROM) 208 devices, flash memory devices, and/or other non-volatile memory coupled to bus 202. The non-volatile memory 208 may store static information and instructions for processor 204. The storage device 210 may include one or more magnetic disk drives, optical disk drives, solid-state disk drives, and/or other mass storage devices coupled to bus 202. The storage device 210 may store information and/or instructions in a persistent manner for processor 204.

The processor 204 may be coupled via bus 202 to the mass spectrometer interface 211. The mass spectrometer interface 211 may operatively couple the computing device 200 and processor 204 to the mass spectrometer 110 and its components. To this end, the mass spectrometer interface 211 may include various I/O and/or networking interfaces. For example, the mass spectrometer interface 211 may include I/O interfaces such as Universal Serial Bus (USB) interfaces, Peripheral Component Interconnect (PCI) interfaces, PCI Express interfaces, Serial Peripheral Interface (SPI) interfaces, FireWire interfaces, etc. Alternatively or additionally, the mass spectrometer interface 211 may include one or more networking interfaces such as Ethernet interfaces, Wi-Fi interfaces, and Bluetooth interfaces.

The processor 204 may be further coupled via bus 202 to a display 212, such as a light emitting diode (LED) or liquid crystal display (LCD). The processor 204 may use the display 212 to present information to a computer user. An input device 214, including alphanumeric and other keys, may be coupled to bus 202. A computer user may utilize the input device to communicate information and command selections to processor 204. The computing device 200 may further include a cursor control 216 coupled to the bus 202. The cursor control 216 may comprise a mouse, a trackball, cursor direction keys, etc., which permit a computer user to select graphical elements or other aspects presented via the display 212. In some aspects, the cursor control 216 may control the movement of a cursor on display 212 used to select such graphical elements or other aspects presented via the display 212. The cursor control 216 typically has two degrees of freedom in two axes, a first axis (e.g., a horizontal axis or x-axis) and a second axis (e.g., a vertical axis or y-axis), that permits the cursor control 216 to move a cursor across a plane of the display 212 and select an x-y position in the plane.

Consistent with certain implementations of the present disclosure, the computing device 200 may operate based on processor 204 executing instructions stored in memory 206. Such instructions may be read into memory 206 from another computer-readable medium, such as storage device 210. Execution of the instructions stored in memory 206 may cause processor 204 to perform various processes described herein. Alternatively, hard-wired circuitry may be used in place of or in combination with software instructions to implement various processes described herein. Thus, implementations of the present disclosure may utilize hardware circuitry and/or software to perform the various processes described herein.

In various aspects, the computing device 200 may be connected to one or more other computing devices across a network to form a networked system. Such other computing devices may be implemented in a manner similar to computing device 200. The network may comprise a private network or a public network such as the Internet. In the networked system, one or more computing devices may store and serve the data to other computing devices. The one or more computing devices 200 that store and serve the data may be referred to as servers, data servers, and/or a data cloud in various cloud-computing scenarios. In some aspects, the one or more computing devices 200 may include one or more web servers that provide other computing devices with web interfaces, web APIs, and/or other access to data and other resources of the one or more computing device. Such computing devices that send and receive data to and from the servers, data servers, and/or the data cloud, regardless of whether via such web servers or web APIs may be referred to as client devices and/or cloud devices.

The term “computer-readable medium” as used herein refers to any media that participates in providing instructions to processor 204 for execution. Such a medium may take many forms, including transitory media (e.g., transmission media) and non-transitory media (e.g., non-volatile media and volatile media). Transmission media may include, for example, coaxial cables, copper wire, fiber optics, the wires that comprise bus 202, and wireless transmissions. Non-volatile media may include, for example, non-volatile storage devices such as those of the non-volatile memory 208 and/or the storage device 210. Similarly, the volatile media may include, for example, volatile storage devices such as those of the volatile memory 206.

Common forms of computer-readable media or computer program products include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer may read.

Various forms of computer-readable media may be involved in carrying one or more sequences of one or more instructions to processor 204 for execution. For example, the instructions may initially be carried on the magnetic disk of a remote computer. The remote computer may load the instructions into its dynamic memory and send the instructions over a communications link. A modem or other network interface local to the computing device 200 may receive the instructions and transfer the received instructions to memory 206 and/or processor 204 via bus 202. The instructions received by memory 206 may optionally be stored in or on storage device 210 either before or after execution by processor 204.

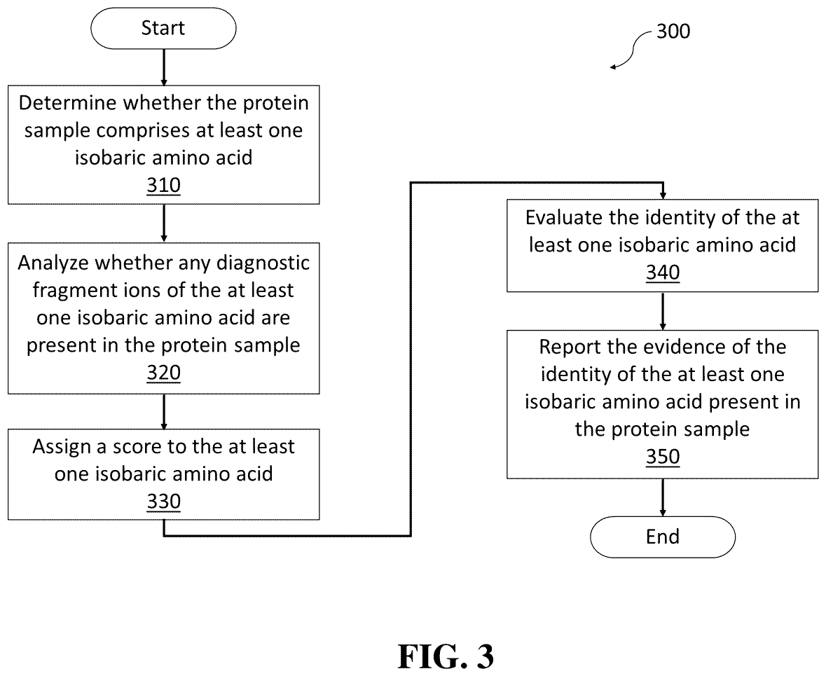

Referring now to FIG. 3, a flowchart depicts an embodiment of an analytical process 300 which may be implemented by the mass analysis system 100 to process an MS/MS data set. As described above, the mass spectrometer 110 may be coupled to one or more computing devices 180, which may receive spectral data from the mass spectrometer 110, analyze the spectral data, and present results of such analysis of the spectral data. For example, the one or more computing devices 180 may process an MS/MS data set that was collected using electron-based dissociation (ExD). As described above, ExD may include, but is not limited to, electron-induced dissociation (EID), electron impact excitation in organics (EIEIO), electron capture dissociation (ECD), and electron transfer dissociation (ETD). The MS/MS data set may be collected from a biological sample, such as a protein sample. In certain aspects, the protein sample may be digested, using, for example, enzymes or chemical methods, to form two or more peptides prior to being analyzed by the mass spectrometer. Thus, the sample analyzed by the mass spectrometer may be a peptide sample. The MS/MS data set may include a spectrum with mass-to-charge ratio (m/z) peaks that correspond to fragment ions.

The processing by the one or more computing devices 180 may include several steps. At 310, the one or more computing devices 180 may determine whether the protein sample comprises at least one isobaric amino acid. As utilized herein, the term “isobaric amino acid” refers to an amino acid that has the same molecular weight as another amino acid. Examples of isobaric amino acids include isoaspartic acid, aspartic acid, isoleucine, and leucine.

The processing may further comprise, at 320, analyzing whether any diagnostic fragment ions of the at least one isobaric amino acid are present in the protein sample. In some aspects, the protein sample may lack isobaric amino acid residues. In other aspects, the protein sample may include a single isobaric amino acid residue. For example, the protein sample may include a single isoaspartic acid residue, a single aspartic acid residue, a single isoleucine residue, or a single leucine residue. In other aspects, the protein sample may include two or more isobaric amino acid residues. For example, in some aspects, each of the two or more isobaric amino acid residues present in the protein sample are the same isobaric amino acid. For example, the protein sample may include two or more isoaspartic acid residues, two or more isoleucine residues, or two or more leucine residues. In other aspects, the protein sample may include two or more isobaric amino acid residues, wherein the two or more isobaric amino acids includes two or more different isobaric amino acids. For example, the two or more isobaric amino acids may include one or more of at least two of the amino acids selected from isoaspartic acid, isoleucine, and leucine.

One of ordinary skill in the art understands that a protein includes a plurality of amino acid residues. A peptide is comparable to a protein, but has a lower molecular weight (i.e., a peptide contains a smaller number of amino acid residues). As discussed above, in some aspects the protein sample analyzed by the present invention may be digested to generate two or more peptide samples prior to analysis by mass analysis system 100. Thus, in some aspects the sample analyzed by the mass spectrometer 110 may be a peptide sample.

Tandem mass spectrometry (i.e., MS/MS) of protein and peptide samples can generate a variety of different types of fragment ions. To this end, the MS/MS data set may include a spectrum of m/z peaks that correspond to different types of fragment ions generated in fragmentation cell 140. Further, as discussed above, the fragmentation cell 140 may be configured to conduct certain fragmentation (also referred to as dissociation) techniques or methodologies. The type of fragmentation technique employed may impact the types of fragment ions generated in fragmentation cell 140 and detected by the ion detector 160.

Certain fragmentation techniques are capable of generating fragment ions that are diagnostic for isobaric amino acids (referred to as “diagnostic fragment ions”). For example, electron-based dissociation (ExD) techniques can generate diagnostic fragmentation ions. As described above, ExD may include, but is not limited to electron-activated dissociation (EAD) techniques such as electron-induced dissociation (EID), electron impact excitation in organics (EIEIO), hot ECD, negative ion ECD (niECD), electron detachment dissociation (EDD), electron capture dissociation (ECD), and electron transfer dissociation (ETD).

The processing may further include, at 330, assigning a score to the at least one isobaric amino acid based on the diagnostic fragment ions present in the MS/MS data set. Assigning a score may be based on various factors. For example, in some aspects, assigning a score may further include assigning independently weighted values for each diagnostic fragment ion. In certain aspects, the value of the score may be based on the number of diagnostic fragment ions present in the MS/MS data set. For example, in certain aspects, the assigned independently weighted value may be higher when a larger number of diagnostic fragment ions are present in the MS/MS data set. In further aspects, the value of the score may be based on the type of diagnostic fragment ions present in the MS/MS data set. For example, the score assigned to a pair of two diagnostic fragment ions that are complementary to each other will be assigned an independently weighted value that is higher than a score assigned to a pair of two diagnostic fragment ions that are not complementary to each other. The term “complementary ion pair” as used herein refers to a pair of fragment ions that have a common bond cleavage along the peptide backbone.

At 340, the processing may further include evaluating the identity of the at least one isobaric amino acid present in the protein sample based on the assigned score.

At 350, the processing may further include reporting the evidence of the identity of the at least one isobaric amino acid present in the protein sample. In certain aspects, the one or more computing devices 180 may display one or more indicators to report the evidence of the identity of the at least one isobaric amino acid present in the protein sample. For example, in certain aspects, the reporting may include displaying a first indicator or a second indicator when one or more diagnostic fragment ions are present in the MS/MS data set. The first indicator may indicate that the score is above a score threshold and the second indicator may indicate that the score is below a score threshold. To this end, various types of indicators may be displayed, such as a color-coded indicator system. For example, the first indicator may be a certain color (e.g., green) to indicate that the score is above the score threshold while the second indicator may be another color (e.g., orange) to indicate that the score is below a score threshold. In certain aspects, a score that is below the score threshold may indicate that manual data analysis is required to determine whether the isobaric amino acid is present in the protein sample. In other aspects, a score that is below the score threshold may indicate that the isobaric amino acid is not present in the protein sample.

In certain aspects, the reporting may further include displaying any diagnostic fragment ions that are present in the MS/MS data set. For example, the one or more computing devices 180 may display any diagnostic fragment ions that are present in the MS/MS data set in a table. In other aspects, the one or more computing devices 180 may display the raw MS/MS spectra to allow for manual evaluation of any diagnostic fragment ions present in the protein sample.

### Diagnostic Fragment Ions

As described above, MS/MS data sets collected by mass spectrometer 110 may be utilized to analyze and report evidence related to the presence or absence of an isobaric amino acid in a protein or peptide sample. For example, the mass analysis system 100 may implement analytical process 300 to process an MS/MS data set. In certain aspects, the mass spectrometer 110 may be coupled to one or more computing devices 180, which may receive spectral data from the mass spectrometer 110, analyze the spectral data, and present results of such analysis of the spectral data.

Table 1 is a summary of diagnostic fragment ions for three isobaric amino acids: isoaspartic acid, aspartic acid, isoleucine, and leucine. One of ordinary skill in the art understands that fragment ions can be denoted with different nomenclature. To this end, Table 1 includes both the nomenclature common in the literature as well as the nomenclature that may be used by a software (e.g., the one or more computing devices 180).

TABLE 1
Summary of Diagnostic Fragment Ions

| Fragment | Fragment | | |
|---|---|---|---|
| Ion | Ion | | Relevant Amino |
| (Literature) | (Software) | Definition | Acid |
| c′ₙ | cₙ | N-terminal peptide backbone | Asp^{a}, isoAsp^{b} |
| | | fragment | |
| z′ₘ | zₘ + 1 | C-terminal peptide backbone | Asp, isoAsp, |
| | | fragment | Leu^{c}, Ile^{d} |
| c′ₙ + 57 | cₙ + 57 | N-terminal peptide backbone | isoAsp |
| | | fragment with side-chain addition | |
| z′ₘ − 57 | zₘ + 1 − 57 | C-terminal peptide backbone | isoAsp |
| | | fragment with side-chain loss | |
| wₘ | wₘ | C-terminal peptide backbone | Ile (wₘ = z′ₘ − 29) |
| | | fragment with side chain loss | Leu (wₘ = z′ₘ − 43) |
| wₘ | w_{mb} | C-terminal peptide backbone | Ile (wₘ = z′ₘ − 15) |
| | | fragment with side chain loss | |

^{a}Asp corresponds to aspartic acid;
^{b}isoAsp corresponds to isoaspartic acid;
^{c}Leu corresponds to leucine;
^{d}Ile corresponds to isoleucine

As discussed above, MS/MS data sets collected by mass spectrometer 110 may be utilized to analyze and report evidence related to the presence or absence of an isobaric amino acid in a protein or peptide sample (e.g., as described in analytical process 300). For example, the one or more computing devices 180 may determine at 310 whether the protein sample comprises at least one isobaric amino acid, wherein the at least one isobaric amino acid includes isoaspartic acid (isoAsp). In certain aspects, the diagnostic fragment ions for isoaspartic acid may include c′ₙ, c′ₙ+57, z′ₘ, and z′ₘ−57. In other aspects, the at least one isobaric amino acid includes isoleucine (Ile). In certain aspects, the diagnostic fragment ions for isoleucine may include z′ₘ, wₘ (z′ₘ−15), and wₘ (z′ₘ−29). In other aspects, the at least one isobaric amino acid includes leucine (Leu). In certain aspects, the diagnostic fragment ions for leucine may include z′ₘ and wₘ (z′m−43).

As described above, the assigned score at 330 may be based on various factors. For example, the assigned score may be based on the presence or absence of certain diagnostic fragment ions, such as those diagnostic fragment ions described above. In certain aspects, the value of the score may further comprise assigning independently weighted values for each diagnostic fragment ion analyzed at 320. In further aspects, the value of the score may be based on the number of fragment ions present in the MS/MS dataset. For example, in certain aspects, a higher weighted value may be assigned when a larger number of diagnostic fragment ions are present in the MS/MS data set. In further aspects, the value of the score may be based on the type of diagnostic fragment ions present in the MS/MS data set. For example, the score assigned to a pair of two diagnostic fragment ion that are complementary to each other will have a higher weight than a score assigned to a pair of two diagnostic fragment ions that are not complementary to each other.

As described above, the diagnostic fragment ions for isoaspartic acid may include c′ₙ, c′ₙ+57, z′ₘ, and z′ₘ−57. Table 2 describes an embodiment wherein the at least one isobaric amino acid determined at 310 includes isoaspartic acid (isoAsp). Table 2 describes possible combinations of these four diagnostic fragment ions for isaspartic acid that may be present in the MS/MS data set. As shown in Table 2, a score may be assigned based on the number of diagnostic fragment ions present in the MS/MS data set, the type of diagnostic fragment ions present in the MS/MS data set, and/or the relationship between the diagnostic fragment ions present in the MS/MS data set (e.g., whether two diagnostic fragment ions are complementary to one another). In certain aspects, the scores may be normalized wherein the maximum score is 100 (i.e., indicating the highest level of confidence that the isobaric amino acid is present in the protein sample).

Table 2 further describes an embodiment including assigned scores and reported evidence of the identity of the at least one isobaric amino acid, wherein the at least one isobaric amino acid includes isoaspartic acid. As described above, the reporting may comprise displaying a first indicator or a second indicator when one or more diagnostic fragment ions are present in the MS/MS data set. The first indicator may indicate that the score is above a score threshold and the second indicator may indicate that the score is below a score threshold. Various types of indicators may be displayed, such as a color-coded indicator system. For example, as shown in Table 2, the first indicator may be a certain color (e.g., green) to indicate that the score is above the score threshold while the second indicator may be another color (e.g., orange) to indicate that the score is below a score threshold.

TABLE 2
Diagnostic Fragment Ions and Scores for isoaspartic Acid

| | # fragment | | | | | | |
|---|---|---|---|---|---|---|---|
| Example | ions | c′ₙ | c′ₙ + 57 | z′ₘ | z′ₘ − 57 | Score | Indicator |
| A | 4 | [symbol] | [symbol] | [symbol] | [symbol] | 100 | Green |
| B | 3 | [symbol] | [symbol] | X | [symbol] | 80 | Green |
| C | 3 | X | [symbol] | [symbol] | [symbol] | 80 | Green |
| D | 3 | [symbol] | X | [symbol] | [symbol] | 60 | Green |
| E | 3 | [symbol] | [symbol] | [symbol] | X | 60 | Green |
| F | 2 | [symbol] | [symbol] | X | X | 60 | Green |
| G | 2 | X | X | [symbol] | [symbol] | 60 | Green |
| H | 2 | X | [symbol] | X | [symbol] | 50 | Green |
| I | 2 | X | [symbol] | [symbol] | X | 20 | Orange |
| J | 2 | [symbol] | X | X | [symbol] | 20 | Orange |
| K | 2 | [symbol] | X | [symbol] | X | N/A | No Color |
| | | | | | | | Indicator |
| L | 1 | X | X | X | [symbol] | 20 | Orange |
| M | 1 | X | [symbol] | X | X | 20 | Orange |
| N | 1 | X | X | [symbol] | X | N/A | No Color |
| | | | | | | | Indicator |
| O | 1 | [symbol] | X | X | X | N/A | No Color |
| | | | | | | | Indicator |

As described above, various factors may determine the value of the assigned score, including the number of diagnostic fragment ions present in the MS/MS data set, the type of diagnostic fragment ions present in the MS/MS data set, and the relationship between two or more ions present in the MS/MS data set. For example, referring to Table 2, example A, where four diagnostic fragment ions are present, is assigned the highest score of 100 compared to other examples. In certain aspects, as discussed above, the assigned score comprises assigning independently weighted values for each diagnostic fragment ion. Further, when at least two diagnostic fragment ions are present in an MS/MS data set, the relationship between those diagnostic fragment ions may be a factor in determining the assigned score. For example, a complementary ion pair may be assigned a higher weight than a non-complementary ion pair. For example, referring to Table 2, z′ₘ and z′ₘ−57 are examples of two diagnostic fragment ions for isoaspartic acid that are complementary to each other. To this end, referring to examples G and H from Table 2, example G is assigned a higher score than example H. While examples G and H both comprise two diagnostic fragment ions, example G is assigned a higher score due to the presence of the complementary ion pair z′ₘ and z′ₘ−57.

As shown in Table 2, examples A-H are examples of combinations of diagnostic fragment ions wherein the assigned score is above a score threshold (indicated by the bold line separating examples H and I in Table 2). As such, these examples may be assigned a “green” indicator to indicate that, based on the diagnostic fragment ions present in the MS/MS data set, there is a high confidence that the isobaric amino acid (i.e., isoaspartic acid in Table 2) is present in the protein sample. As shown in Table 2, examples I, J, L, and M represent combinations of diagnostic fragment ions wherein the assigned score is below a score threshold. As such, these examples may be assigned an “orange” indicator. In certain aspects, when the score is below a score threshold, the one or more computing devices 180 may provide the MS/MS data set in a form suitable for manual analysis for the presence or absence of the one or more isobaric amino acids. For example, the one or more computing devices 180 may provide the raw MS/MS spectra. In other aspects, the one or more computing devices 180 may provide a table of m/z peaks present in the MS/MS data set.

In certain aspects, an MS/MS data set will not be assigned a score for one or more isobaric amino acids. In these aspects, based on the absence of certain diagnostic fragment ions, there is a high confidence that the one or more isobaric amino acids is not present in the protein sample. Referring to Table 2, examples of these MS/MS data sets are indicated in examples K, N, and O (with a score of “N/A” and an indicator of “do nothing”).

Tables 3 and 4 similarly describe embodiments wherein the at least one isobaric amino acid determined at 310 includes other isobaric amino acids. As described above, the diagnostic fragment ions for isoleucine (Ile) may include z′ₘ, wₘ (z′ₘ−15), and wₘ (z′ₘ−29). Table 3 describes an embodiment wherein the at least one isobaric amino acid determined at 310 includes isoleucine.

TABLE 3
Diagnostic Fragment Ions and Scores for Isoleucine

| | # fragment | | wₘ | wₘ | | |
|---|---|---|---|---|---|---|
| Example | ions | z′ₘ | (z′ₘ − 29) | (z′ₘ − 15) | Score | Colour |
| A | 3 | [symbol] | [symbol] | [symbol] | 100 | Green |
| B | 2 | [symbol] | [symbol] | X | 90 | Green |
| C | 2 | X | [symbol] | [symbol] | 70 | Green |
| D | 2 | [symbol] | X | [symbol] | 60 | Orange |
| E | 1 | X | [symbol] | X | 40 | Orange |
| F | 1 | X | X | [symbol] | 20 | Orange |
| G | 1 | [symbol] | X | X | N/A | No Color |
| | | | | | | Indicator |

As described above, the diagnostic fragment ions for leucine (Leu) may include z′ₘ and wₘ (z′ₘ−43). Table 4 describes an embodiment wherein the at least one isobaric amino acid determined at 310 includes leucine.

TABLE 4
Diagnostic Fragment Ions and Scores for Leucine.

| | # fragment | | | | |
|---|---|---|---|---|---|
| Example | ions | z′ₘ | wₘ (z′ₘ − 43) | Score | Colour |
| A | 2 | [symbol] | [symbol] | 100 | Green |
| B | 1 | X | [symbol] | 50 | Orange |
| C | 1 | [symbol] | X | N/A | No Color |
| | | | | | Indicator |

### Isobaric Amino Acid Differentiation in Experimental Workflows

In certain aspects, the methods described herein for isobaric amino acid differentiation may be incorporated into various types of experimental workflows. For example, in certain aspects, the isobaric amino acid differentiation may be incorporated into a peptide mapping workflow.

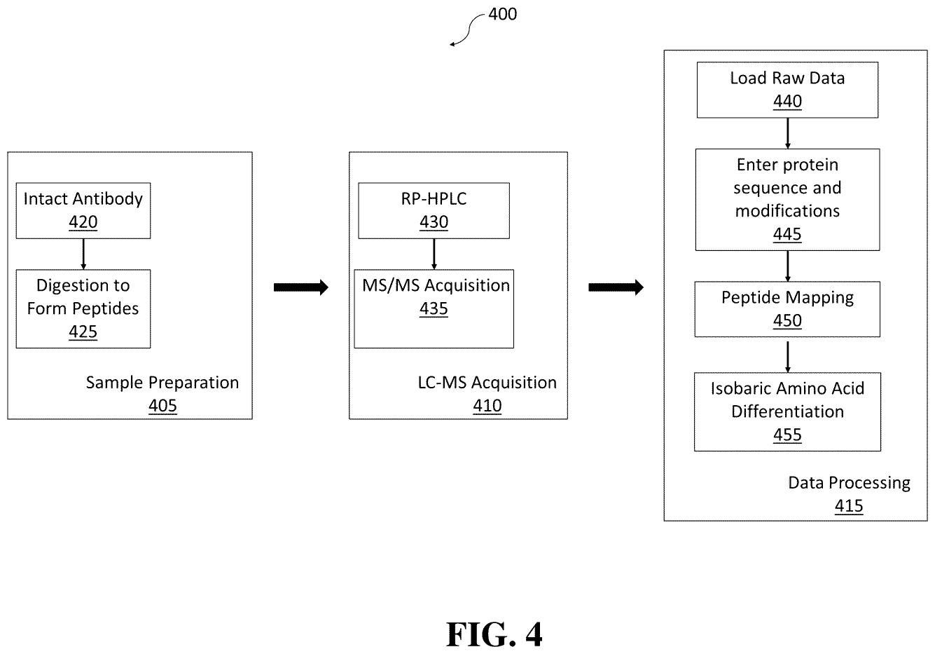

FIG. 4 is a flowchart of an embodiment of a peptide mapping workflow 400 that incorporates isobaric amino acid differentiation. A peptide mapping workflow 400 may include three steps: sample preparation 405, LC-MS acquisition 410 (i.e., liquid chromatography coupled to a mass spectrometer), and data processing 415. The sample preparation 405 may include providing an intact antibody 420 and subjecting the intact antibody to digestion to form peptides 425. Following sample preparation 405, the LC-MS acquisition 410 may include reversed-phase high-performance liquid chromatography (RP-HPLC) 430 followed by MS/MS acquisition 435. The MS/MS acquisition 435 may be collected using an ExD fragmentation technique, such as EAD. The final data processing step 415 may include loading the raw data 440, entering the protein sequence and modifications 445, peptide mapping 450, and isobaric amino acid differentiation 455. The isobaric amino acid differentiation 455 may include any of the aspects and embodiments for methods of isobaric amino acid differentiation described herein.

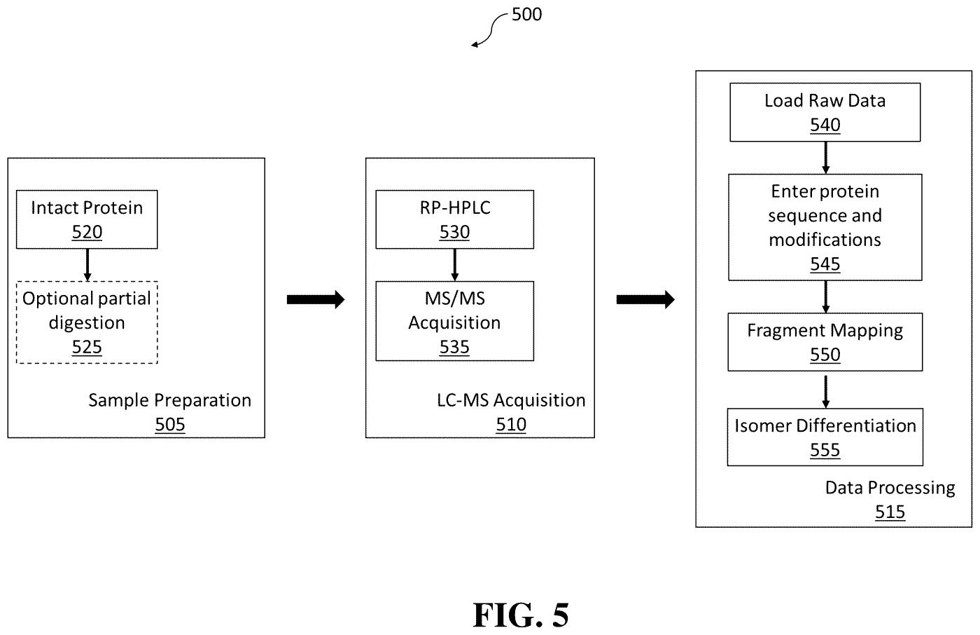

In other aspects, the methods described herein for isobaric amino acid differentiation may be incorporated into a middle-down workflow. FIG. 5 is a flowchart of an embodiment of a middle-down workflow 500 that incorporates isobaric amino acid differentiation. A middle-down workflow may include three steps: sample preparation 505, LC-MS acquisition 510, and data processing 515. The sample preparation 505 may include providing an intact protein 520 and optionally subjecting the intact antibody to partial digestion 525. Following sample preparation 505, the LC-MS acquisition may include RP-HPLC 530 followed by MS/MS acquisition 535. The MS/MS acquisition 535 may be collected using an ExD fragmentation technique. The final data processing step 515 may include loading the raw data 540, entering the sequence and modifications 545, fragment mapping 550, and isobaric amino acid differentiation 555. The isobaric amino acid differentiation 555 may include any of the aspects and embodiments for methods of isobaric amino acid differentiation described herein.

While the present disclosure has described certain aspects, various changes may be made and equivalents may be substituted without departing from the scope of the present invention. In addition, many modifications may be made to adapt a particular situation or material to the teachings of the present disclosure without departing from the scope of the appended claims. Therefore, the present disclosure is not limited to the particular embodiment disclosed, but includes all aspects falling within the scope of the appended claims.

1. A computer-implemented method, comprising:
processing, with one or more computing devices, an electron-based dissociation (ExD) MS/MS data set of a protein sample, the MS/MS data set comprising a spectrum with mass-to-charge ratio (m/z) peaks that correspond to fragment ions, wherein the processing further comprises:
determining whether the protein sample comprises at least one isobaric amino acid;
analyzing whether any diagnostic fragment ions of the at least one isobaric amino acid are present in the protein sample;
assigning a score to the at least one isobaric amino acid based on the diagnostic fragment ions present in the MS/MS data set;
evaluating an identity of the at least one isobaric amino acid present in the protein sample based on the assigned score; and
reporting the evidence of the identity of the at least one isobaric amino acid present in the protein sample.

2. The computer-implemented method of claim 1, wherein the assigning a score further comprises assigning independently weighted values for each diagnostic fragmentation.

3. The computer-implemented method of claim 1, wherein the assigning a score further comprises assigning a higher weight when a larger number of diagnostic fragment ions are present.

4. The computer-implemented method of claim 1, wherein the score further comprises assigning higher weight to a complementary ion pair than a non-complementary ion pair.

5. The computer-implemented method of claim 1, wherein the reporting comprises displaying a first indicator or a second indicator when one or more diagnostic fragment ions are present in the MS/MS data set, wherein the first indicator indicates the score is above a score threshold and the second indicator indicates the score is below a score threshold.

6. The computer-implemented method of claim 5, wherein the reporting further comprises displaying any diagnostic fragment ions that are present in the MS/MS data set.

7. The computer-implemented method of claim 1, wherein the at least one isobaric amino acid includes isoaspartic acid.

8. The computer-implemented method of claim 7, wherein the diagnostic fragment ions for isoaspartic acid are c′ₙ, c′ₙ+57, z′ₘ, and z′ₘ−57.

9. The computer-implemented method of claim 8, wherein the z′ₘ and z′ₘ−57 diagnostic fragment ions are a first complementary ion pair and c′ₙ and c′ₙ+57 diagnostic fragment ions are a second complementary ion pair.

10. The computer-implemented method of claim 1, wherein the at least one isobaric amino acid includes isoleucine.

11. The computer-implemented method of claim 10, wherein the diagnostic fragment ions for isoleucine are z′ₘ, wₘ (z′ₘ−15), and wₘ (z′ₘ−29).

12. The computer-implemented method of claim 1, wherein the at least one isobaric amino acid includes leucine.

13. The computer-implemented method of claim 12, wherein the diagnostic fragment ions for leucine are z′ₘ and wₘ (z′ₘ−43).

14. The computer-implemented method of claim 1, wherein the computer-implemented method is incorporated into a peptide mapping workflow.

15. One or more non-transitory computer-readable storage media comprising instructions, which when executed by one or more computing devices, causes the one or more computing devices to:
process, with one or more computing devices, an electron-based dissociation (ExD) MS/MS data set of a protein sample, the MS/MS data set comprising a spectrum with mass-to-charge ratio (m/z) peaks that correspond to fragment ions, wherein the processing further comprises:
determining whether the protein sample comprises at least one isobaric amino acid;
analyzing whether any diagnostic fragment ions of the at least one isobaric amino acid are present in the protein sample;
assigning a score to the at least one isobaric amino acid based on the diagnostic fragment ions present in the MS/MS data set;
evaluating an identity of the at least one isobaric amino acid present in the protein sample based on the assigned score; and
reporting the evidence of the identity of the at least one isobaric amino acid present in the protein sample.
